(19) United States
(12) Patent Application Publication
(10) Pub. No.: US 2009/0259218 A1
(43) Pub. Date: Oct. 15, 2009

(54) APPARATUS AND METHODS FOR SKIN TREATMENT

(75) Inventors: Ahmed Bitter, London (GB); Antony Grant, Gloucestershire (GB); Alex Wolski, Gloucester (GB); Desmond Mills, Gloucestershire (GB); Kevin Herbert, Gluocestershire (GB)

Correspondence Address: YOUNG & THOMPSON, 209 Madison Street, Suite 500, ALEXANDRIA, VA, 22314, US

(73) Assignee: THE DEZAC GROUP LIMITED, Cheltenham, Gloucestershire (GB)

(21) Appl. No.: 12/306,171

(22) PCT Filed: Jun. 21, 2007
(86) PCT No.: PCT/GB07/02305

§ 371 Date: Dec. 22, 2008

(30) Foreign Application Priority Data
Jun. 22, 2006 (GB) 0612391.3

(51) Int. Cl. A61B 18/20

(52) U.S. Cl. 606/9

(57) ABSTRACT

A hair treatment device for the treatment of the human or animal skin by laser radiation to prevent or reduce hair growth, which device includes a laser radiation source for emitting a laser radiation beam; beam deflecting elements for deflecting the radiation beam across the skin, the deflecting elements including a lens through which the beam of laser radiation passes, and elements for moving the lens to effect deflection of the beam.

This invention relates to apparatus and methods for skin treatment and in particular, but not exclusively, to the treatment of human or animal skin using laser radiation to effect a cosmetic and/or therapeutic treatment, for example hair removal.

It is already known to use laser treatment for hair removal. Laser radiation is directed towards the skin with the radiation being absorbed in the hair follicle and on the skin surface. The wavelength of the laser radiation is selected so as to be absorbed by melanin in the follicle so that the hair is heated to a temperature which causes it to stop growth. Although incidence of the laser radiation on the skin can also cause local skin heating, the heating of the hair follicle is much more acute.

Laser removal targeting one hair at a time with a narrowly focused laser beam is relatively inefficient and time consuming. To improve the time for treatment, many modern laser hair removal devices perform hair removal by focusing a number of lasers onto an area so as to treat a number of hair follicles simultaneously. However, the laser emitter is usually the most expensive component of a laser hair remover and so provision of several laser emitters in a device makes it expensive to manufacture and therefore unsuitable for the general domestic market.

Accordingly there is a need for a hair removal device that is cost effective to manufacture but which still allows a region somewhat larger than a single beam spot to be treated.

A further consideration is that the life of generally available laser emitters may be reduced if their routine operation requires repeated switching on and off of the emitter during treatment. It is therefore desirable, though not essential, for the hair device not to require repeated switching whilst treatment is being carried out.

Accordingly, in one particular aspect this invention provides a hair treatment device for the treatment of the human or animal skin by laser radiation to prevent or reduce hair growth, which device comprises:

a laser radiation source for emitting a laser radiation beam;

beam deflecting means for deflecting said radiation beam across the skin, said deflecting means comprising an optical element such as e.g. a lens through which said beam of laser radiation passes, and means for effecting relative movement of said optical element and said source to effect deflection of said beam.

It will of course be appreciated that, instead of having the laser radiation source relatively fixed with the lens moving relative to the source, the opposite arrangement is also possible whereby the lens is kept relatively stationary whilst the laser is moved. Still further, it would be possible to effective relative movement by moving both the lens and the laser source, for example if there was a constraint on the movement of either due to the size of the device.

In another aspect, this invention provides a device for the treatment of the human or animal skin, which comprises:

a radiation source for emitting a radiation beam;

movement means for moving said radiation beam in a predetermined pattern across the skin.

The treatment effected thereby may be exclusively cosmetic treatment, exclusively therapeutic treatment or a mixture thereof. For example, the treatment may comprise one or more of:

removal of hair

removal of tattoos or other skin pigmentation

treatment of visible capillaries such as port wine stains or surface veins, rosacea and similar discolourations

treatment to reduce the appearance of cellulite.

Preferably, said radiation source comprises a laser radiation source. The laser radiation source may take many forms, but in one arrangement may be a laser diode. The laser radiation source is preferably selected to emit radiation at a predetermined wavelength selected according to the nature of the treatment. In the case of laser treatment for hair removal the laser radiation source preferably has a wavelength of between 750 nm and 850 nm and more preferably about 808 nm. Again, the fluence of the laser radiation will be selected according to the nature of the particular treatment but for hair removal the fluence of the laser at the target site is preferably greater than 15 J/cm², and more preferably greater than about 20 J/cm².

In order to increase the operating life of the device, it is preferred that the radiation source emits a generally continuous beam once activated. However we do not exclude arrangements where the radiation source emits a pulsed beam.

The movement means may take many forms such as a mirror or other reflector, but it is preferably in the form of a refracting means and, more specifically, a lens, mounted for movement, with there being drive means for effecting relative movement of the refracting means relative to the laser to deflect the beam axis. In one arrangement, the drive means is operable to shift the lens linearly in at least one direction generally transverse to the axis of the radiation beam emitted from the radiation source. More particularly, the drive means is preferably operable to shift said lens linearly in two generally orthogonal directions relative to the axis of the radiation beam. In this manner, taking a coordinate system in which the radiation beam is the Z axis, the beam may be deflected in the X and Y directions by corresponding shifting of the lens.

In another arrangement, the drive means may be operable to tilt the lens about at least one axis transverse to the beam axis and, more preferably, about two generally orthogonal axes.

The drive means may take many different forms but is preferably electromagnetic comprising a permanent a magnet and a coil with a current being passed through the coil to exert movement. However, other types of operation such as a piezo-electric device, an electric motor or a mechanical movement or a combination of these. In a mechanical arrangement the movement may be affected by providing a roller or other suitable element on the end of the device, which rotates as the device is drawn across the skin, the rotary motion of the roller being transmitted via a suitable transmission mechanism to effect said drive.

The device preferably includes control means operable to control the drive means to deflect said radiation beam. The control means may be operable to cause said beam to execute a scan pattern with a generally continuously moving scan spot. Alternatively, the control means may cause the beam to execute a scan pattern with discrete movements of a scan spot interspersed with dwell periods during which the spot is generally stationary. The extent of movement between dwell periods and/or the length of the dwell periods in a scan may be adjusted in accordance with the extent of deflection of the beam from its equilibrium position, so as to compensate for an increase or decrease in scan spot size as it moves.

In one arrangement, the control means is operable to dither the scan spot between selected regions so that selected regions receive multiple sequential exposures.

Whilst the invention has been described above it extends to any inventive combination as set out above or in the following description or claims.

The invention may be performed in various ways, and an embodiment thereof will now be described by way of example only, reference being made to the accompanying drawings, in which:

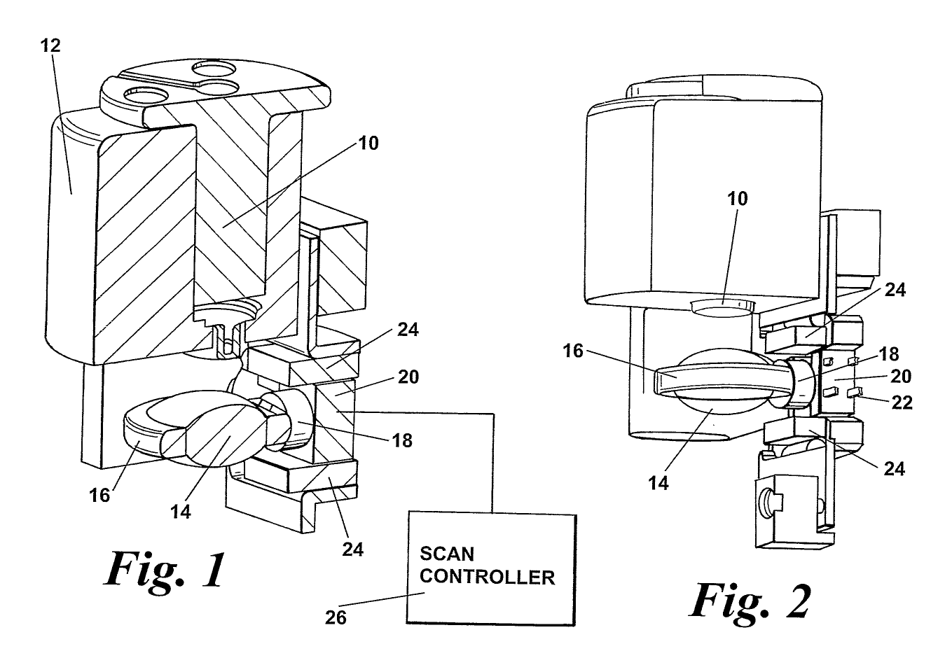

FIG. 1 is a perspective part cutaway view of a laser and deflector assembly for a hair treatment device in accordance with this invention;

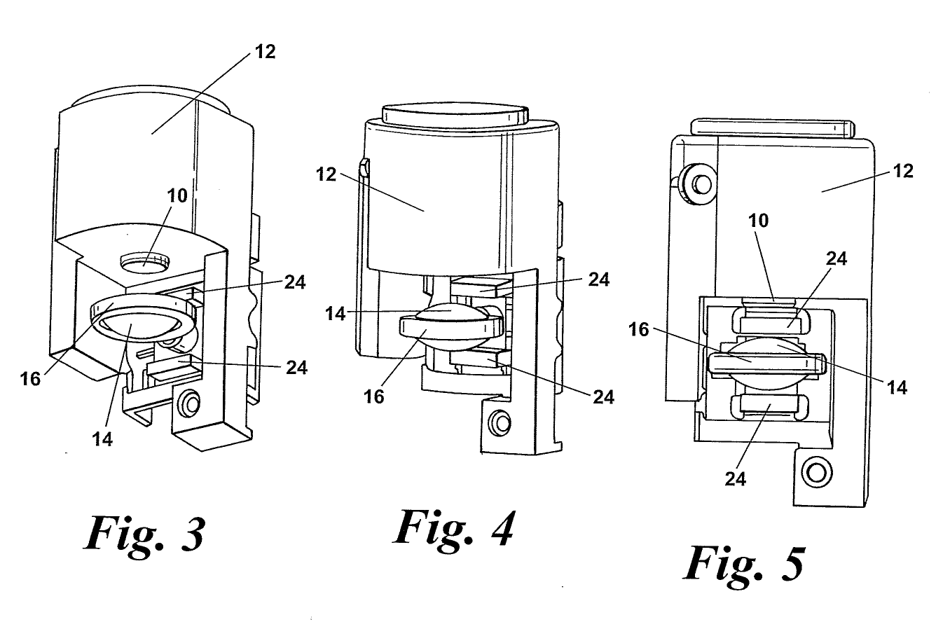

FIGS. 2 to 5 are further views of the assembly of FIG. 1;

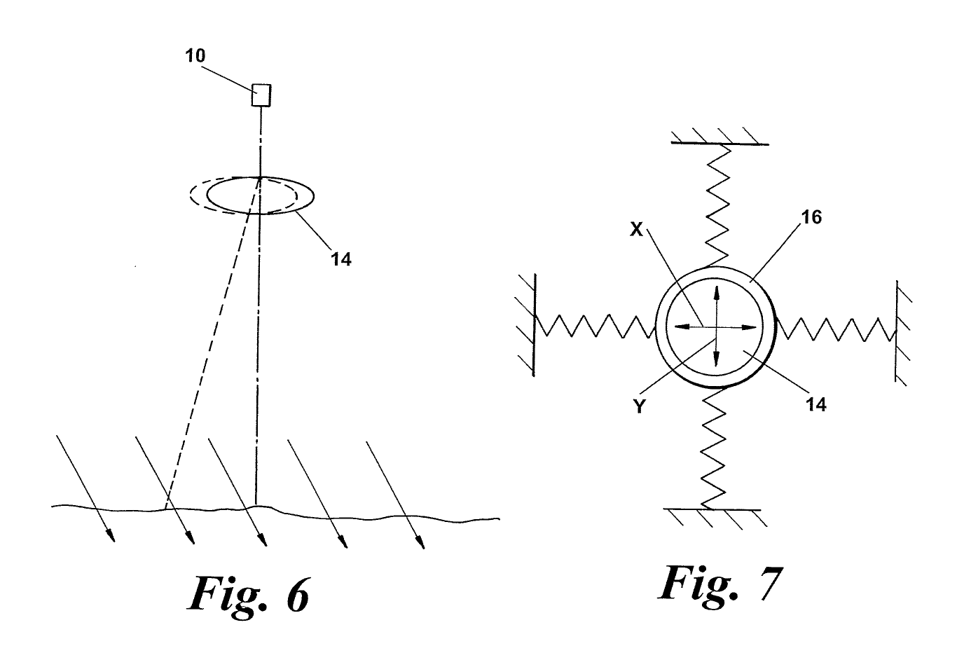

FIG. 6 is a schematic view of the optical arrangement showing movement of the lens to deflect the laser beam, and

FIG. 7 is a schematic view showing the mounting of the lens.

The embodiment illustrated in the Figures discloses an apparatus in which a single laser beam is moved across the surface of the skin to be treated so as to sequentially target and destroy hair follicles. The apparatus consists of a laser emitter, a focussing element (here a lens) which ensures that the beam delivers targeted energy to the target site, and a drive. In the various embodiments, the beam may be continuous so that it ‘sweeps’ the skin surface or it may be pulsed so that it moves in discrete steps. The movement of the beam may be controlled by one or more of the following: movement of a lens (as in the illustrated embodiment to be described below); movement of a mirror; and movement of the laser emitter. The movement may be achieved by means of an induction motor (as in the illustrated embodiment); a piezo-electric means, an electric motor mechanism with a transmission drive; a mechanical drive or any combination of the above.

In the embodiment described below, a laser emitter is used which passes the laser beam through a moveable lens. As the beam passes through the lens, it is subject to differing optical properties which result in the exiting laser beam passing through a different angle depending on which part of the lens it entered. Although the use of mirrors is not excluded, the use of a lens is preferred because reflective mirrors are generally expensive to manufacture; they are highly subject to optical degradation due to shock, moisture, heat and debris within the device, and moreover the commercial implementation requires use of a lens to focus the beam and so an existing component can be used thereby reducing the number of additional components that might otherwise compromise the reliability of the device.

Referring now to the illustrated embodiment, the device consists of a laser emitter 10, mounted in a suitable heat sink block 12, and a focusing lens 14 housed within a moveable support ring 16. The support ring is connected via a stub 18 to a printed circuit board (PCB) 20. The PCB is resiliently mounted for movement in two perpendicular directions in the plane of the lens by means of suitable X and Y flexural mountings 22 (only one set of which is seen in FIG. 2). The PCB has two drive coils, an X drive coil and a Y drive coil (neither shown). The PCB 20 is supported between two permanent magnets 24. The X and Y coils on the PCB 20 and the permanent magnets 24 therefore operate similarly to the voice coils in a loudspeaker. The X direction coil moves the lens from left to right when viewed as in FIG. 5 and the Y direction coil moves the lens in and out as seen in that view. The applied voltage (negative or positive) to the coils determines the direction and amount of movement of the lens in either the X and Y direction. The voltages to the coils are controlled by a scan controller 26 shown schematically in FIG. 1.

In use, the controller 26 passes current through the X plane coil in PCB 20 so as to cause the lens to move in the X direction and this has the effect of causing the beam to track along the X plane of the skin as shown schematically in FIG. 6. When the beam is at a predetermined limit, the Y plane coil is also energised causing the beam to move in the Y direction. The X plane coil may then be de-energised causing the beam to retraces its movement, this time at a different Y position. Thus with coordinated control, the laser beam can be tracked across the skin in both the X and Y planes to scan a shaped treatment area.

In a preferred embodiment, the laser moves in steps equivalent to the diameter of the laser beam or scan spot, so as to uniformly treat an area of skin. Once the beam reaches the furthest extent of the X direction, the Y coil is energised and the beam moved by one laser diameter in the Y direction so as to provide a stepped form of raster scan. Scan movements may be pre-programmed or may be as a result of a sensory feedback from the skin surface indicating that the treatment in that area is complete. This could be by means of a suitable detector (such as an IR detector) detecting the temperature and determining that treatment in that area is complete.

Where the controller applies a stepped scan pattern, the controller may adjust the magnitude of the steps, decreasing with increasing angle of incidence, to take into account the divergence of the laser beam as the angle to the skin's normal becomes greater; it will be noted that the beam tends to diverge as this angle increases.

In another scan pattern, the controller moves the laser to scan it rapidly between two adjacent target sites. In this manner, the target sites experience a series of repeated exposures, somewhat similar to a fixed pulsed beam. By adjusting the duration and delay of the exposures the target site may receive sufficient repeated applications of energy to cause the hair to progressively heat up and prevent growth, whilst the temperature of the skin does not appreciably rise, because of the differing heating times and heating absorption/emission characteristics of the hairs and the surrounding tissue. Due to the relative melanin contents, hair absorbs laser energy more rapidly than the surrounding skin tissue and thus heats quicker. Furthermore, due to the geometry and anatomy of the hair compared to the surrounding tissue it loses heat slower. Thus by repeatedly subjecting the target area to discrete exposures to radiation it is possible to heat the hair follicle whilst keeping the surrounding skin at a nominal temperature. In the above embodiment, the effect similar to a pulse beam with radiation, is actually achieved by rapidly scanning between two adjacent target sites whilst the laser is continually powered in the avoidance of rapidly switching the laser on and off, thereby prolonging its operational life.

1. A hair treatment device for the treatment of the human or animal skin by laser radiation to prevent or reduce hair growth, which device comprises:
a laser radiation source for emitting a laser radiation beam;
beam deflecting means for deflecting said radiation beam across the skin, said deflecting means comprising an optical element through which said beam of laser radiation passes, and means for effecting relative movement of said optical element and said source to effect deflection of said beam.

2. A device for the treatment of the human or animal skin, which comprises:
a radiation source for emitting a beam of radiation;
movement means for moving said radiation beam in a predetermined pattern across the skin.

3. A device according to claim 1, wherein said treatment comprises cosmetic treatment.

4. A device according to claim 3, wherein said treatment comprises therapeutic treatment.

5. A device according to claim 3, wherein said cosmetic treatment is effective for the removal of hair.

6. A device according to claim 3, wherein said treatment is effective for the removal of tattoos or other pigmentation.

7. A device according to claim 3, wherein said treatment is effective for treatment of blood vessels.

8. A device according to claim 3, wherein said treatment is effective for the treatment of cellulite.

9. A device according to claim 1, wherein said source of radiation comprises a source of laser radiation.

10. A device according to claim 9, wherein said source of laser radiation comprises a laser diode.

11. A device according to claim 8, wherein said laser radiation has a wavelength of between 750 nm and 850 nm.

12. A device according to claim 9, wherein said laser beam has a fluence of greater than 15 J/cm².

13. A device according to claim 1, wherein said radiation source emits a generally continuous beam.

14. A device according to claim 1, wherein said radiation source emits a pulsed beam.

15. A device according to claim 1, wherein said movement means comprises a lens mounted for movement and drive means for moving said lens to deflect the beam axis.

16. A device according to claim 15, wherein said drive means is operable to shift said lens linearly in at least one direction generally transverse to the axis of the beam emitted from said radiation source.

17. A device according to claim 16, wherein said drive means is operable to shift said lens linearly in two generally orthogonal directions relative to the axis of said beam.

18. A device according to claim 15, wherein said drive means is operable to tilt said lens about at least one axis generally transverse to the axis of the beam emitted from said radiation source.

19. A device according to claim 18, wherein said drive means is operable to tilt said lens about two generally orthogonal axes relative to the axis of said beam.

20. A device according to claim 1, wherein said movement means comprises a deflector mounted for movement and drive means for moving said deflector to deflect the beam axis.

21. A device according to claim 20, wherein said drive means is operable to tilt said deflector about at least one axis generally transverse to the axis of the beam emitted from said radiation source.

22. A device according to claim 1, wherein said movement means comprises an emitter mounted for movement and drive means for moving said emitter to translate the beam axis.

23. A device according to claim 22, wherein said drive means is operable to shift said emitter linearly in at least one direction generally transverse to the axis of the beam emitted from said radiation source.

24. A device according to claim 23, wherein said drive means is operable to shift said emitter linearly in two generally orthogonal directions relating to the axis of said beam.

25. A device according to claim 22, wherein said drive means is operable to tilt said emitter about at least one axis generally transverse to the axis of the beam emitted from said radiation source.

26. A device according to claim 15, further including control means operable to control said drive means to deflect said beam.

27. A device according to claim 26, wherein said control means is operable to cause said beam to execute a scan pattern with a generally continuously moving spot.

28. A device according to claim 26, wherein said control means is operable to cause said scan to execute a scan pattern with discrete movements of a scan spot interspersed with dwell periods.

29. A device according to claim 28, wherein said control means is operable to adjust the length of the dwell period, or the extent of each discrete movement, in accordance with the magnitude of the beam deflection.

30. A device according to claim 28, wherein said control means is operable to dither the scan spot between selected regions.

31. A method of skin treatment, wherein a source of radiation generates a beam which passes via a deflecting means to be incident on the skin, wherein said deflecting means moves said beam across the skin in a predetermined pattern.
